(19) United States
(12) Plant Patent Application Publication
(10) Pub. No.: US 2020/0396880 P1
(43) Pub. Date: Dec. 17, 2020

(54) Grape plant named 'Juneaudor precoce'

(50) ;

(71) Applicant: Ronald Juneau, Pont-Rouge (CA)

(72) Inventor: Ronald Juneau, Pont-Rouge (CA)

(21) Appl. No.: 16/873,714

(22) Filed: Jun. 10, 2020

(30) Foreign Application Priority Data
Jun. 11, 2019 (CA) 19-9941

(51) Int. Cl. A01H 6/88

(52) U.S. Cl. PLT/207

(57) ABSTRACT

The invention is a new and distinct variety of Grape plant named ‘Juneaudor précoce’ that is characterized by its grape clusters with shoulders that are loose in density, its very early fruit maturity period, its vigorous growth habit with high fruit productivity, its good fruit set, its grapes that have thick skins, and its grapes that produce wine that is rich in aroma, juicy, vinous with a fruity taste.

### CROSS-REFERENCE TO A RELATED APPLICATION

This application claims priority to a Canadian plant breeders' rights application filed on Jun. 6, 2019, application No. 19-9941, under 35 U.S.C. 119(f), the entire contents of which is incorporated by reference herein.

Botanical classification: Vitis hybrid.

Variety denomination: ‘Juneaudor précoce’.

### BACKGROUND OF THE INVENTION

The present invention relates to a new and distinct cultivar of grape plant botanically of hybrid origin and known as Vitis ‘Juneaudor précoce’, referred to hereafter by its cultivar name, ‘Juneaudor précoce’.

The new cultivar was derived from a controlled breeding program conducted by the Inventor in Portneuf County, Québec, Canada. The objective of the breeding program is to develop new cold hardy grape varieties with reliable winter hardiness in Québec combined with good winemaking characteristics.

The new cultivar arose from open pollination in 2002 of an unnamed and unpatented proprietary plant in the Inventor's breeding program, reference no. “27 Auger” as the female parentT. The male parent is therefore unknown. The new Vitis was selected as a single unique plant in 2016 from the resulting seedlings.

Asexual propagation of the new cultivar was first accomplished by hardwood stem cuttings in 2012 by the Inventor in St-Ubalde, Québec, Canada. Asexual propagation of the new cultivar by hardwood stem cuttings has determined that the characteristics are stable and true to type in successive generations.

### SUMMARY OF THE INVENTION

The following traits have been repeatedly observed and represent the characteristics of the new cultivar. These attributes in combination distinguish ‘Juneaudor précoce’ as a new and unique cultivar of Vitis.
1. ‘Juneaudor précoce’ exhibits grape clusters with shoulders that are loose in density.
2. ‘Juneaudor précoce’ exhibits a very early fruit maturity period; 3 weeks before the grape varieties grown under the same conditions by the Inventor.
3. ‘Juneaudor précoce’ exhibits a vigorous growth habit with high fruit productivity.
4. ‘Juneaudor précoce’ exhibits grapes that are gold in color at full maturity.
5. ‘Juneaudor précoce’ exhibits good fruit set.
6. ‘Juneaudor précoce’ exhibits grapes that have thick skins.
7. ‘Juneaudor précoce’ exhibits grapes that produce wine that is rich in aroma, juicy, vinous with a fruity taste.

The female parent plant of ‘Juneaudor précoce’ differs from ‘Juneaudor précoce’ in having leaves that are different in form and appearance, grape clusters that are different in shape and fruits that are different in color and size and mature later. ‘Juneaudor précoce’ can also be compared to the Vitis cultivars ‘Adalmiina’ (not patented) and ‘Louise Swenson’ (not patented). ‘Adalmiina’ and ‘Louise Swenson’ are both similar to ‘Juneaudor précoce’ in having fruit that is gold in color at maturity and similar berries and grape cluster characteristics. ‘Adalmiina’ and ‘Louise Swenson’ both differ from ‘Juneaudor précoce’ in having leaves that are different in appearance and form, berry maturity date that is much later, and much less cold hardiness, different berry flavor and different vine buds and young shoots.

### BRIEF DESCRIPTION OF THE DRAWINGS

The accompanying colored photographs illustrate the overall appearance and distinct characteristics of the new grape cultivar. The photographs were taken of five-year-old plants of the new cultivar as grown outdoors in a field in Saint-Ubalde, Québec, Canada.

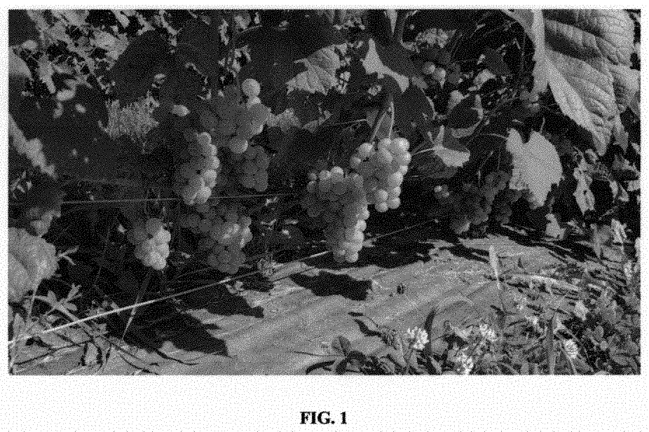

The photograph in FIG. 1 provides a side view of the plant habit of ‘Juneaudor précoce’ with fruit clusters.

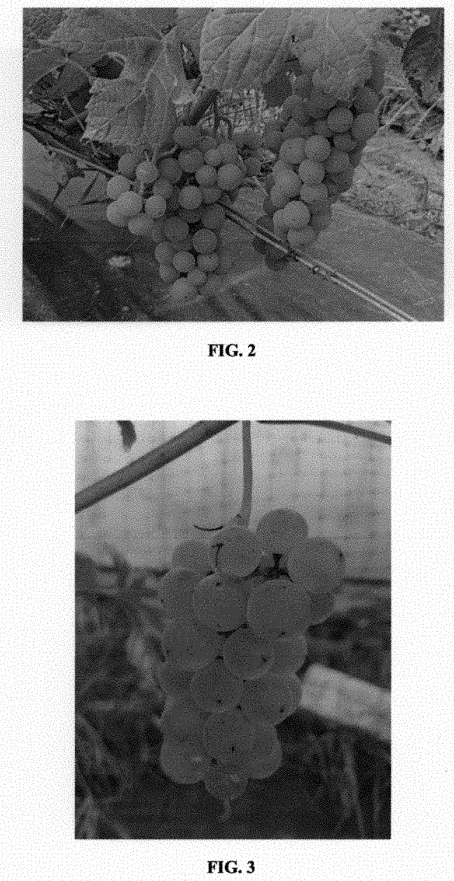

The photograph in FIG. 2 provides a close-up view of the immature fruit of ‘Juneaudor précoce’.

The photograph in FIG. 3 provides a close-up view of the mature fruit of ‘Juneaudor précoce’. The colors in the photographs are as close as possible with digital photography techniques available, the color values cited in the detailed botanical description accurately describe the colors of the new cultivar of grape.

### DETAILED BOTANICAL DESCRIPTION OF THE PLANT

The following is a detailed description of plants five years in age as grown outdoors in a trial field in Saint-Ubalde, Québec, Canada. The phenotype of the new cultivar may vary with variations in environmental, climatic, and cultural conditions, as it has not been tested under all possible environmental conditions. The color determination is in accordance with the 2015 Colour Chart of The Royal Horticultural Society, London, England, except where general color terms of ordinary dictionary significance are used.
General description:
   Blooming period.—Typically commences bloom on June 15^{th} in the County of Portneuf, Québec.
   Plant type.—Perennial fruit producing vine.
   Plant habit.—Open and orderly.
   Height and spread.—An average of 1.2 m in height and 220 cm in width.
   Cold hardiness.—At least in U.S.D.A. Zone 4a.
   Diseases and pests.—Good resistance to downy mildew (Plasmopara viticola), powdery mildew (Uncinula necator), and black rot (Guignardia bidwellii), no resistance or susceptibility to pests has been observed.
   Propagation.—Hardwood stem cuttings.
   Root description.—Fibrous and fine.
   Root development.—An average of 21 days for root initiation with a young plants produced from a rooted cutting produced in 45 to 60 days.
   Growth rate.—Vigorous.

Additional Detailed botanical data will be produced by preliminary amendment.

It is claimed:

1. A new and distinct cultivar of Grape plant named ‘Juneaudor précoce’ as herein illustrated and described.
